(19) United States
(12) Patent Application Publication
(10) Pub. No.: US 2023/0321402 A1
(43) Pub. Date: Oct. 12, 2023

(54) Line Draw Optimized Integrated Catheter Stabilization and Securement System

(71) Applicant: Becton, Dickinson and Company, Franklin Lakes, NJ (US)

(72) Inventors: Jonathan Karl Burkholz, Salt Lake City, UT (US); John M. Lackey, West Valley City, UT (US); Dorien Gemma Butter, Millcreek, UT (US)

(21) Appl. No.: 18/128,640

(22) Filed: Mar. 30, 2023

Related U.S. Application Data
(60) Provisional application No. 63/328,042, filed on Apr. 6, 2022.

(51) Int. Cl. A61M 25/02

(52) U.S. Cl. CPC A61M 25/02; A61M 2025/0253

(57) ABSTRACT

Provided herein is a stabilization device for a catheter, including a base defining a base perimeter, the base having a top surface and a bottom surface, a support surface arranged on the top surface of the base, the support surface having a wall extending away from the top surface of the base and defining a support surface perimeter, the support surface perimeter having substantially the same shape as a catheter adapter, and a dressing defining a dressing perimeter, the dressing configured to cover the base and the support surface.

### CROSS-REFERENCE TO RELATED APPLICATION

The present application claims priority to U.S. Provisional Application Ser. No. 63/328,042, entitled “Line Draw Optimized Integrated Catheter Stabilization and Securement System”, filed Apr. 6, 2022, the entire disclosure of which is hereby incorporated by reference in its entirety.

### BACKGROUND OF THE INVENTION

### Field of the Invention

Provided herein are devices and systems for use in vascular access, and, in particular, stabilization devices and vascular access systems including such devices.

### Description of Related Art

During dwell, a peripheral intravenous catheter (PIVC) may become occluded with thrombus or fibrin, preventing the ability to draw blood through the PIVC. Line draw devices are meant to pass through the catheter and open up a fluid path for blood draw. One challenge with these devices is successfully navigating through the s-shaped profile the catheter takes as it enters the vein. The catheter tubing may kink slightly at the bends of the s-curve (FIG. 2), preventing the line draw device from successfully passing though.

During insertion of a PIVC into a patient, the angle at which the catheter is inserted is typically around 30°, depending on a number of factors including the patient's vasculature, vein depth, and clinician technique. Once the catheter has been successfully inserted into the vein, the catheter adapter will be secured to the patient using adhesive tape or dressings. When securing the catheter adapter, the dressing is placed over the catheter adapter and the surrounding skin such that the catheter adapter lies roughly parallel to the skin. Because the catheter was inserted at a steeper angle relative to the skin, the tubing exiting the catheter adapter must transition from the plane of the adapter down into the vein, thus potentially kinking or deforming the tubing enough that a line draw device would be unable to pass through.

Proper securement of a PIVC is a critical step to ensure the success of line draw devices. Accordingly, there is a need in the art for a stabilization and securement device and dressing system to provide improved overall blood draw success, line draw device advancement reliability, and reduced risk of catheter complications resulting from additional access and use of the PIVC for blood collection.

### SUMMARY OF THE INVENTION

Provided herein is a stabilization device for a catheter, including a base defining a base perimeter, the base having a top surface and a bottom surface, a support surface arranged on the top surface of the base, the support surface having a wall extending away from the top surface of the base and defining a support surface perimeter, the support surface perimeter having substantially the same shape as a catheter adapter, and a dressing defining a dressing perimeter, the dressing configured to cover the base and the support surface.

Also provided herein is a stabilized catheter set, including a catheter assembly having a catheter adapter having a distal end, a proximal end, a lumen arranged between and in fluid communication with the distal end and the proximal end, a catheter arranged at the distal end of the catheter adapter and in fluid communication with the lumen, a stabilization device as described herein.

### BRIEF DESCRIPTION OF THE DRAWINGS

FIG. 1 is a perspective view of a non-limiting embodiment of a catheter adapter useful with a stabilization device as described herein;

FIG. 2 is a partial cross-sectional view of a conventional catheter adapter and catheter, showing the catheter inserted into a vein of a patient;

FIGS. 3A and 3B are perspective and top views of a non-limiting embodiment of a stabilized catheter set as described herein;

FIGS. 4A and 4B are perspective and top views of a non-limiting embodiment of a stabilized catheter set as described herein;

FIGS. 5A and 5B are perspective and top views of a non-limiting embodiment of a stabilized catheter set as described herein;

FIG. 6 is a perspective view of a non-limiting embodiment of a stabilized catheter set as described herein;

FIG. 7 is a perspective view of a non-limiting embodiment of a stabilized catheter set as described herein;

FIGS. 8A and 8B are perspective views of a non-limiting embodiment of a stabilized catheter set as described herein; and

FIGS. 9A and 9B are perspective views of a non-limiting embodiment of a stabilized catheter set as described herein.

### DESCRIPTION OF THE INVENTION

The following description is provided to enable those skilled in the art to make and use the described embodiments contemplated for carrying out the invention. Various modifications, equivalents, variations, and alternatives, however, will remain readily apparent to those skilled in the art. Any and all such modifications, variations, equivalents, and alternatives are intended to fall within the spirit and scope of the present invention.

For purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawing figures. However, it is to be understood that the invention may assume various alternative variations, except where expressly specified to the contrary. It is also to be understood that the specific devices illustrated in the attached drawings, and described in the following specification, are simply exemplary embodiments of the invention. Hence, specific dimensions and other physical characteristics related to the embodiments disclosed herein are not to be considered as limiting.

It should be understood that any numerical range recited herein is intended to include all values and sub-ranges subsumed therein. For example, a range of “1 to 10” is intended to include all sub-ranges between (and including) the recited minimum value of 1 and the recited maximum value of 10, that is, having a minimum value equal to or greater than 1 and a maximum value of equal to or less than 10.

Provided herein is a stabilization device for a catheter assembly. Stabilization devices as described herein may be used to provide a secure site of attachment of a catheter adapter to a site of a venipuncture, and may provide an optimal angle of entry of a catheter into the vasculature of a patient, reducing or eliminating kinking of medical devices that are passed through the catheter into the patient.

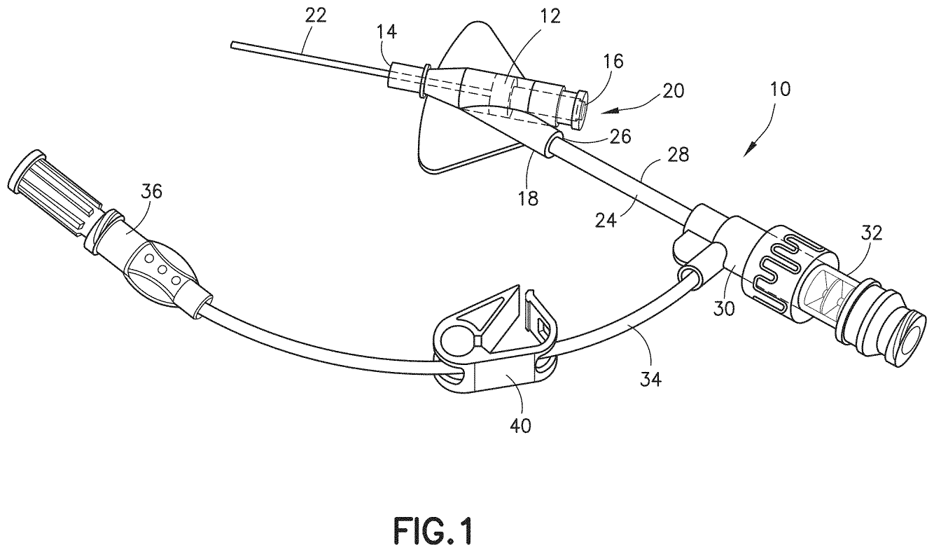

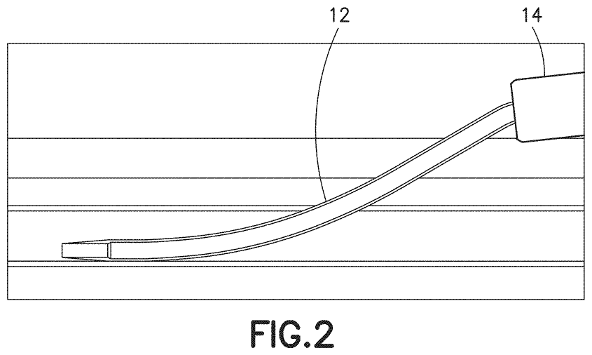

Referring now to FIG. 1, shown is a non-limiting embodiment of a catheter assembly 10 useful with stabilization devices as described herein below. Cather assembly 10 may include a catheter adapter 12, which may include a distal end 14 and a proximal end 16. In some embodiments, the catheter adapter 12 may include an additional port 18. In some embodiments, port 18 may disposed between the distal end 14 and the proximal end 16. In some embodiments, port 18 may be disposed at proximal end 16. In some embodiments, the first catheter adapter 12 may include a first lumen 20 extending through the distal end 14 and the proximal end 16. First lumen 20 may be sealed at proximal end 16 of catheter adapter 12.

In some non-limiting embodiments or aspects, the catheter assembly 10 may include a catheter 22 extending from the distal end 14. In some embodiments, the first catheter 22 may include a peripheral intravenous catheter, a midline catheter, or a peripherally-inserted central catheter. Catheter 22 may be formed of any suitable material and may be of any useful length, as known to those of skill in the art. In some non-limiting embodiments or aspects, the catheter assembly 10 may include a first fluid conduit 24 extending from the port 18. First fluid conduit 24 may be formed of any suitable material known to those of skill in the art, and may have a distal end 26 and a proximal end 28, and first fluid conduit 24 may be coupled, at distal end 26 thereof, to port 18. In some non-limiting embodiments or aspects, a connector 30 may be coupled to a proximal end 28 of first fluid conduit 24. Connector 30 may be a t-connector (e.g., one side port arranged at a 90 degree angle relative to a longitudinal axis of connector 30), a y-connector (e.g., one side port arranged at a 25, 60, or 75 degree angle relative to a longitudinal axis of connector 30), or any other type of connector known in the art, and may include a second lumen therethrough, having any number of branches suitable for the type of connector.

In some non-limiting embodiments or aspects, catheter assembly 10 may include an extension set (integrated into or removably coupleable to catheter adapter 12, connector 30, and/or needleless access connector 32) including a second fluid conduit, such as second fluid conduit 34. Extension sets are known to those of skill in the art and are commercially available from, for example, Becton, Dickinson and Company under the tradenames MAXPLUS, MAXZERO, and NEUTRACLEAR. Needleless access connectors 32 are known to those of skill in the art and are commercially available from, for example, Becton, Dickinson and Company under the tradenames SMARTSITE and Q-SYTE. In some non-limiting embodiments or aspects, second fluid conduit 34 may include a luer connection 36 at an end thereof. In some non-limiting embodiments or aspects, second fluid conduit 34 may include a clamp 40, to allow for occlusion of second fluid conduit 34. Clamp 40 and second fluid conduit 34 may be formed of any suitable materials known to those of skill in the art. In non-limiting embodiments, second lumen (e.g., within connector 30) has an inner diameter that is substantially equivalent to an inner diameter of first fluid conduit 24 and/or second fluid conduit 34.

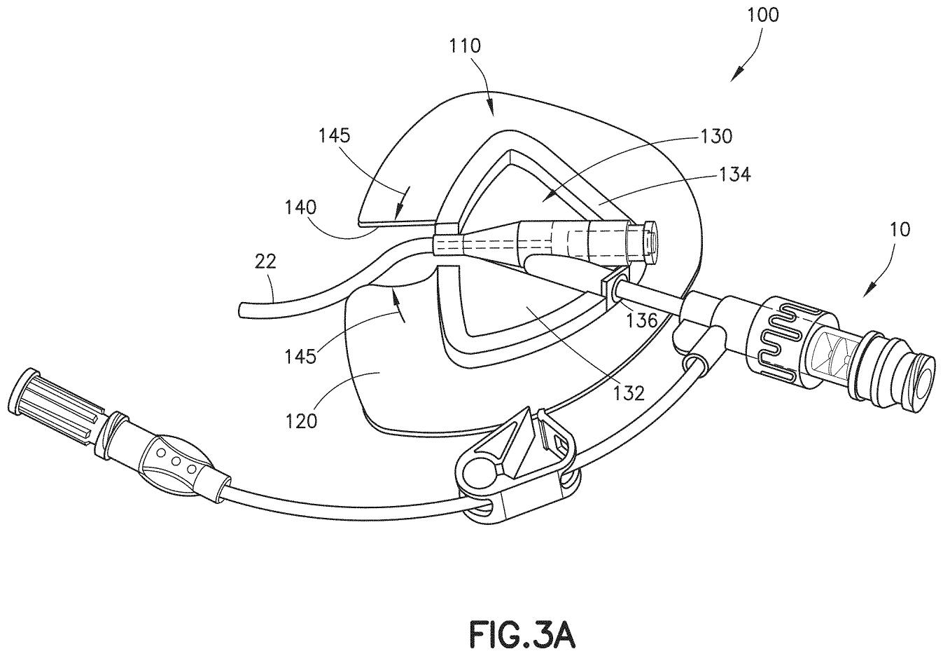

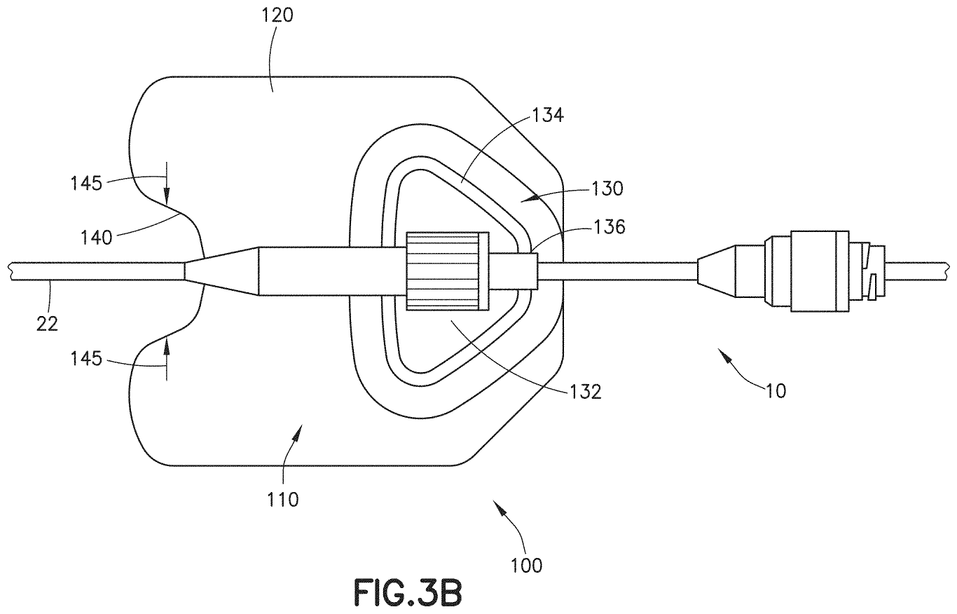

Turning to FIGS. 3A and 3B, shown are non-limiting embodiments of a stabilized catheter system 100, including a catheter assembly 10 and a stabilization device 110. FIG. 3A shows a catheter adapter 12 with port 18 as a side port, extending away from catheter adapter 12 at an angle relative to a longitudinal axis define by the distal end 14 and proximal end 16 of the catheter adapter 12. FIG. 3B shows a catheter adapter 12 with port 18 arranged at proximal end 16 thereof. Those of skill will appreciate that stabilization devices 110 described herein may be modified to accommodate catheter adapters with any arrangement of port(s). Stabilization device 110 may include a base dressing 120 having a top surface, a bottom surface configured to contact skin of a patient onto which base dressing 120 is applied, and a perimeter defining a base perimeter. Base dressing 120 may be formed of any suitable material, for example woven and non-woven fabrics, films, polymeric materials, and like materials known to those of skill in the art. Base dressing 120 may include, on bottom surface thereof, an adhesive, to reversibly attach base dressing 120 to skin of a patient. Suitable adhesives are known to those of skill in the art. As shown in the accompanying figures, base dressing 120 may include a cutout 140 to accommodate a catheter 22 at the site of insertion into the patient's skin. Cutout 140 may also accommodate any additional adhesives or antimicrobial compositions applied to the site of insertion of catheter 22 into the patient's skin.

Base dressing 120 may further include a first support 130. First support 130 may include a first support surface 132 configured to receive a catheter adapter thereon. First support surface 132 may be defined by a wall 134 extending away from the top surface of the base dressing 120 (e.g., away from the skin of a patient). The combination of first support surface 132 and wall 134 may define a pocket in which a catheter adapter 12, and/or other component of a catheter assembly 10, is received. Wall 134 may define a support surface perimeter that substantially matches catheter adapter 12, and, optionally, one or more additional components of catheter assembly 10. Wall 134 may define a support surface perimeter that, in embodiments, may be smaller than a base perimeter. First support surface 132 may be formed of any useful material. In non-limiting embodiments, first support surface 132 is formed of a foam, such as an open or closed cell foam, or other resilient material that can be compressed. In non-limiting embodiments, first support surface 132 includes an adhesive thereon, for securing a catheter adapter 12 in place. In non-limiting embodiments, first support surface 132 is configured such that a catheter adapter 12 received thereon is arranged at an angle relative to skin of a patient onto which stabilization device 110 is applied. In non-limiting embodiments, first support surface 132 is configured such that a catheter adapter 12 is arranged at an angle of between about 3 degrees and about 10 degrees (all values and subranges therebetween inclusive), optionally between about 6 degrees and about 10 degrees, optionally about 6 degrees, relative to (away from) skin of a patient onto which stabilization device 110 is applied. In non-limiting embodiments, support surface 132 may be adjustable to provide a different angle of catheter adapter 12 relative to the patient's skin, depending on whether catheter 22 is not being used, is being used to deliver a medicament, and/or is being used to draw blood from the patient's vasculature.

Wall 134 may be formed of any suitable material, including rigid foams. Wall 134 may include one or more openings 136 therein, to accommodate catheter 22, any port(s) 18 included on catheter adapter 12, and/or any fluid conduits 24, 34 extending from catheter adapter 12. One or more openings 136 in wall 134 may include an adhesive to secure components of catheter assembly 10 thereto.

With continuing reference to FIGS. 3A and 3B, base dressing 120 may include one or more alignment markers 145. Alignment marker(s) 145 may be any suitable indicia that are visible to a medical professional applying stabilization device 110 to skin of a patient. Alignment marker(s) 145 may be arranged on one or both sides of cutout 140. Alignment marker(s) 145 may indicate the position on the patient's skin where the catheter 22 is inserted, and may be configured at a predetermined location on base dressing 120, optimized to reduce and/or eliminate an s-curve in catheter 22.

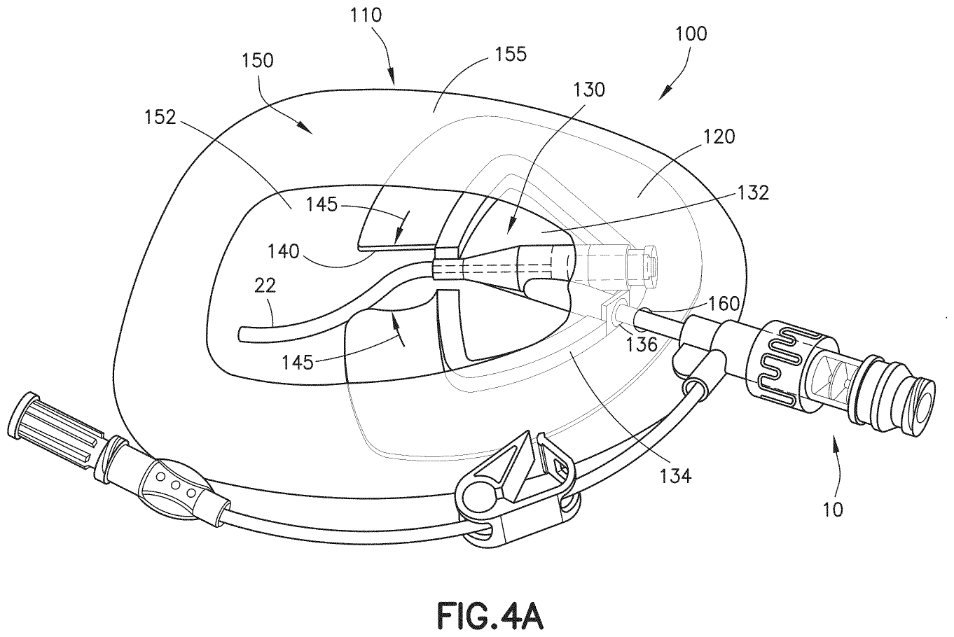

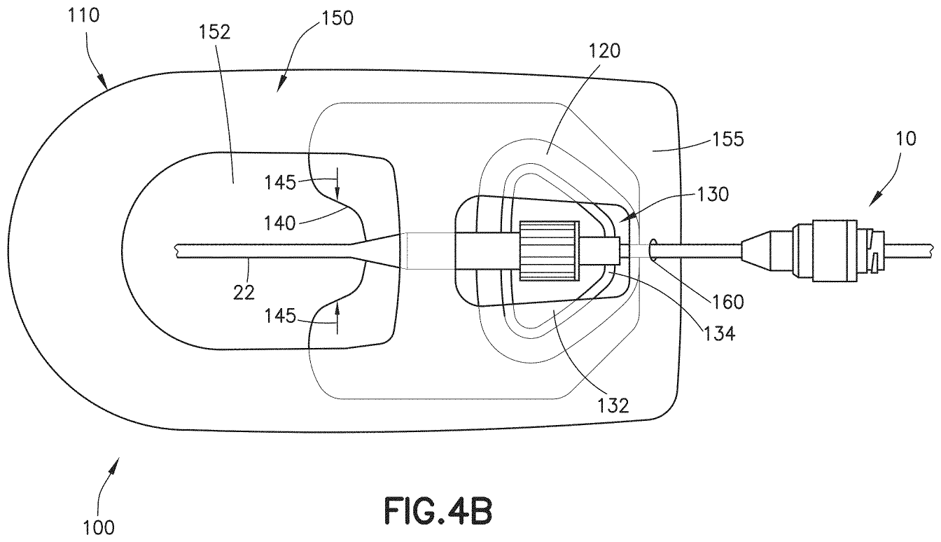

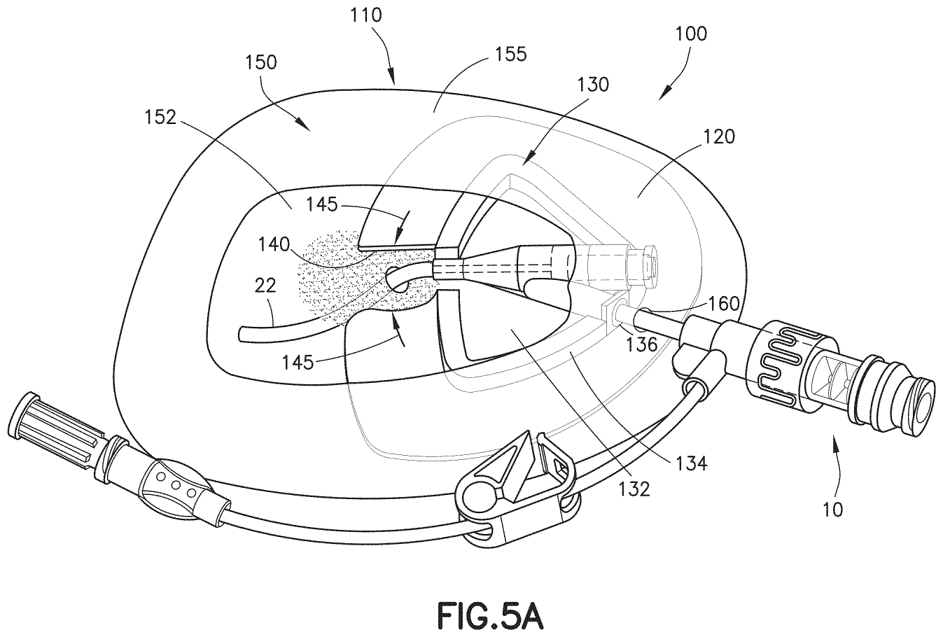

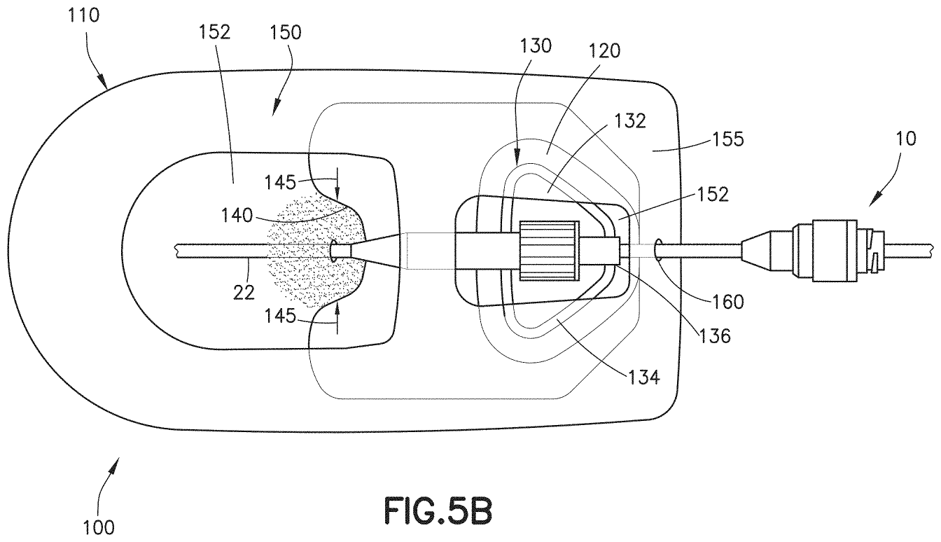

Turning to FIGS. 4A-5B, stabilization device 110 may also include top dressing 150. Top dressing 150 may be formed of any suitable material, for example polymeric materials known to those of skill in the art such as polyurethanes and polyethylenes, woven and non-woven fabrics, and/or combinations thereof, and may include adhesives (for example to releasably adhere top dressing 150 to base dressing 120 and/or a patient's skin), antimicrobials, and other suitable additives known to those of skill in the art. Suitable top dressings 150 are also available commercially, for example under the tradename TEGADERM from 3M (St. Paul, MN). In non-limiting embodiments, at least a portion 152 of top dressing 150 is at least partially transparent to allow for visualization of catheter adapter 12 and/or other components of catheter assembly 10. In non-limiting embodiments, top dressing 150 has a perimeter that is larger than that of the perimeter of base dressing 120. In non-limiting embodiments, top dressing 150 includes a fabric portion 155. In non-limiting embodiments, top dressing 150 includes one or more openings and/or extensions 160 to allow a connector 30, port 18, and/or fluid conduit 24, 34 attached to or extending from catheter adapter 12 to pass through top dressing 150, and/or to allow top dressing 150 to be wrapped about a connector 30, port 18, and/or fluid conduit 24, 34 attached to or extending from catheter adapter 12.

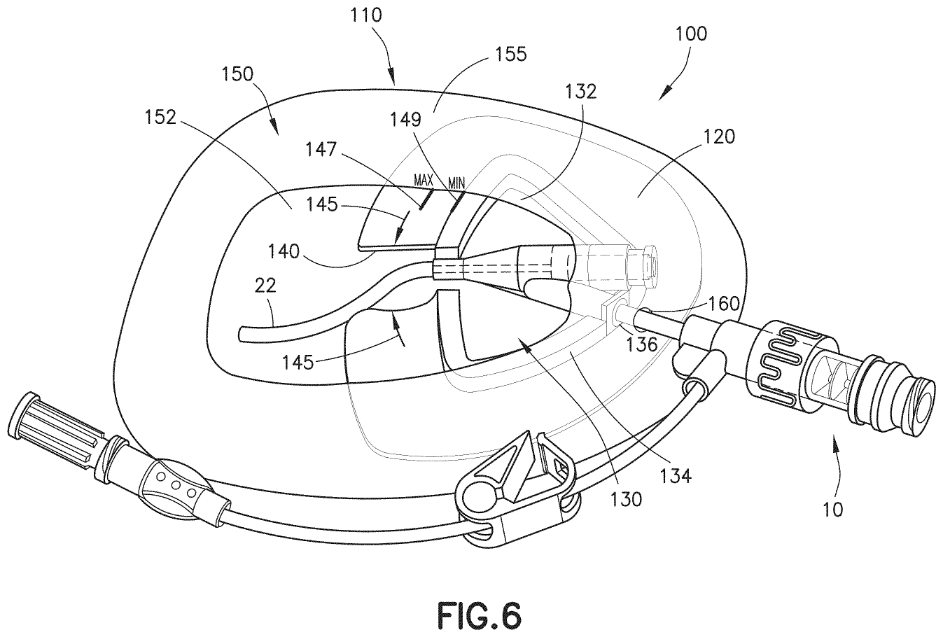

Turning to FIG. 6, in non-limiting embodiments, stabilization device 110 may include, on base dressing 120, one or more additional markers. Such additional markers may be in addition to, or in place of, alignment marker(s) 145 discussed herein. Such additional markers may include maximum 147 and minimum 149 distance markers, which may correspond to a maximum and minimum distance that base dressing 120 can be positioned from the insertion site of catheter 22 sufficient to maintain adequate sterility, insertion depth/length, and/or minimize any s-curve in catheter 22. Maximum and minimum 147, 149 distance markers may correspond to a distance between distal end 14 of catheter adapter 12 and insertion site of catheter 22. In non-limiting embodiments, minimum distance marker 149 indicates a distance of about 0.0 mm, optionally about 0.50 mm, and maximum distance marker 147 indicates a distance of about 2.50 mm, from between distal end 14 of catheter adapter 12 and insertion site of catheter 22. Maximum and minimum 147, 149 distance markers may be any suitable indicia, and may be of different colors or the same color, and may be arranged on one or both sides of cutout 140.

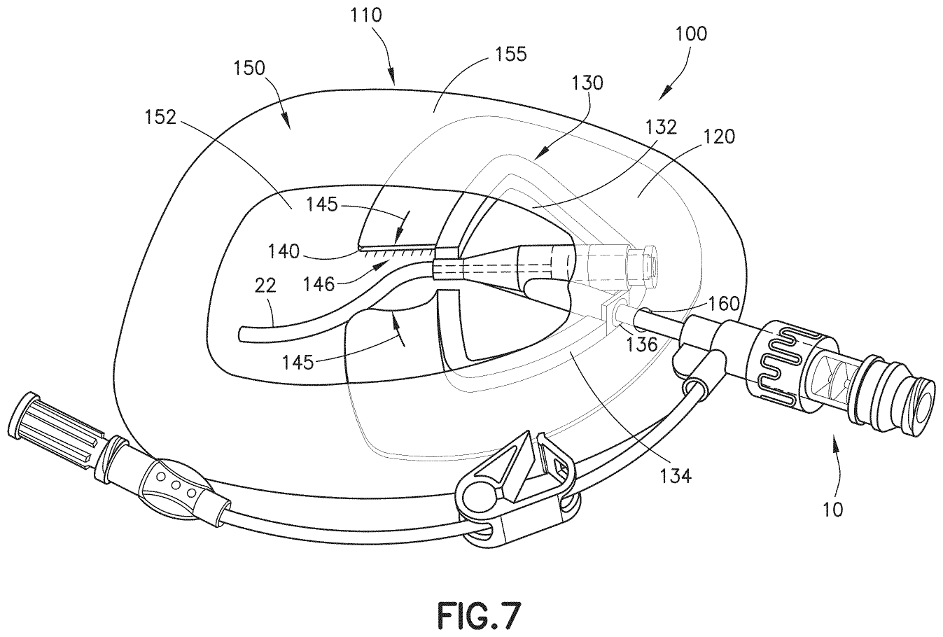

Turning to FIG. 7, in non-limiting embodiments, such additional markers may include one or more displacement markers 146. Displacement marker(s) 146 may be any suitable indicia, and may be of different colors or the same color, and may be arranged on one or both sides of cutout 140. In non-limiting embodiments, displacement marker(s) 146 are arranged relative to alignment marker(s) 145, and function to provide an indication to a medical professional of any displacement of stabilization device 110, for example displacement of base dressing 120, relative to the site of insertion of catheter 22. This can aid a medical professional in determining whether stabilization device 110 has moved an unacceptable difference (e.g., raising issues of continued sterility or of an unacceptable extent of s-curve of catheter 22), such that it should be removed and/or reapplied to the patient's skin.

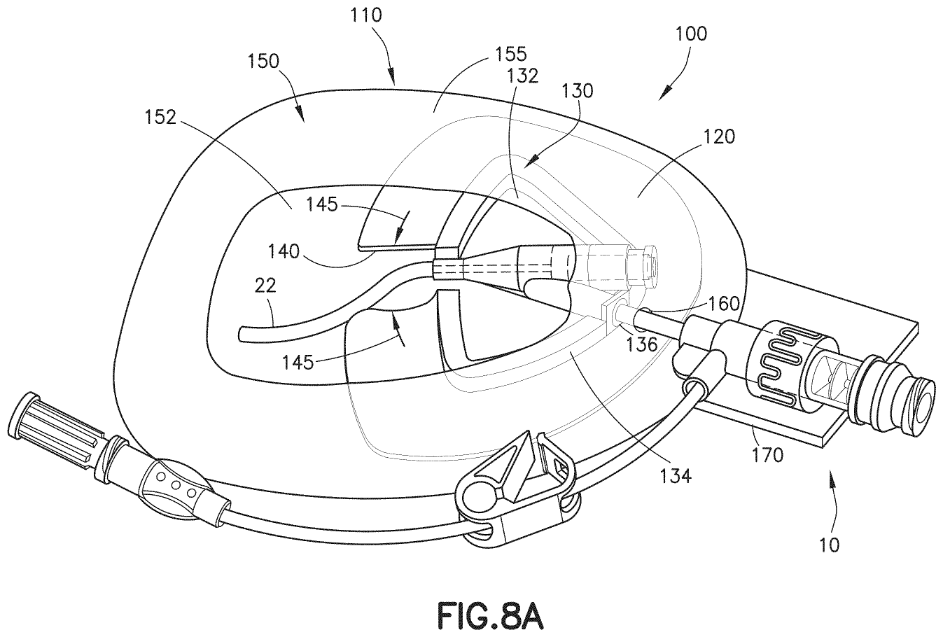

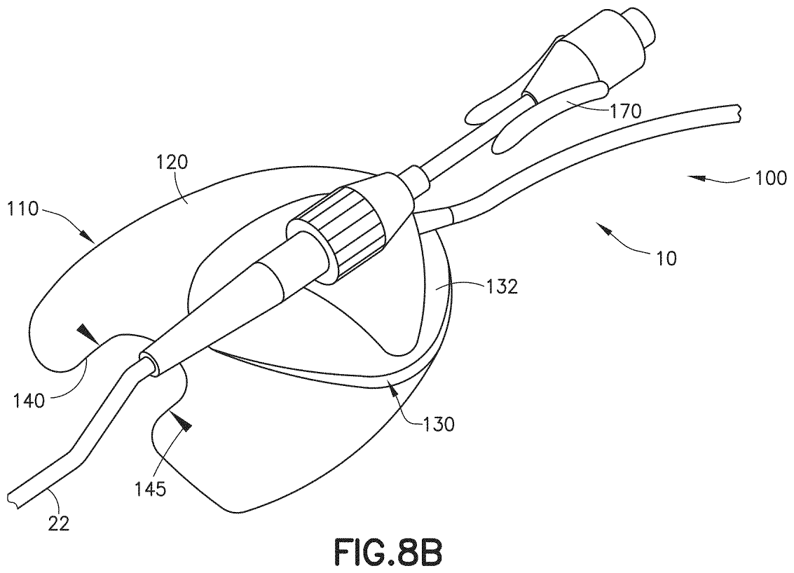

Turning to FIGS. 8A and 8B, in non-limiting embodiments, stabilization device can further include a second support 170. Second support 170 can, like first support 130, be formed of any suitable material, such as a resilient material, a foam material, or the like. Second support 170 may be configured to interact with a connector 30, such as a t-connector, y-connector, and/or needless access connector 32, or other medical component, that is included as part of or otherwise coupled to one or more components of catheter assembly 10. For example, as shown in FIG. 8A (t- or y-connector 30) and 8B (needleless access connector 32) may be supported by second support 170, which may function to reduce pressure of the medical component on the patient's skin and/or secure the component to the patient. Second support 170 may include any suitable features to secure the connector 30 or other component, such as one or more pockets, ridges, or the like. Second support 170 may include an adhesive on a lower surface thereof, to releasably adhere second support 170 to the patient's skin.

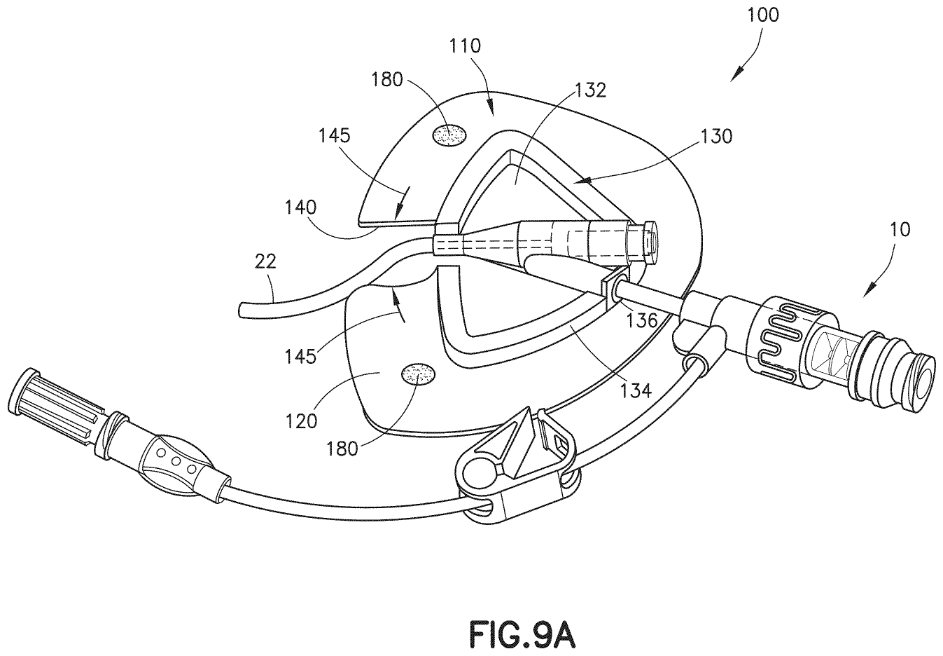

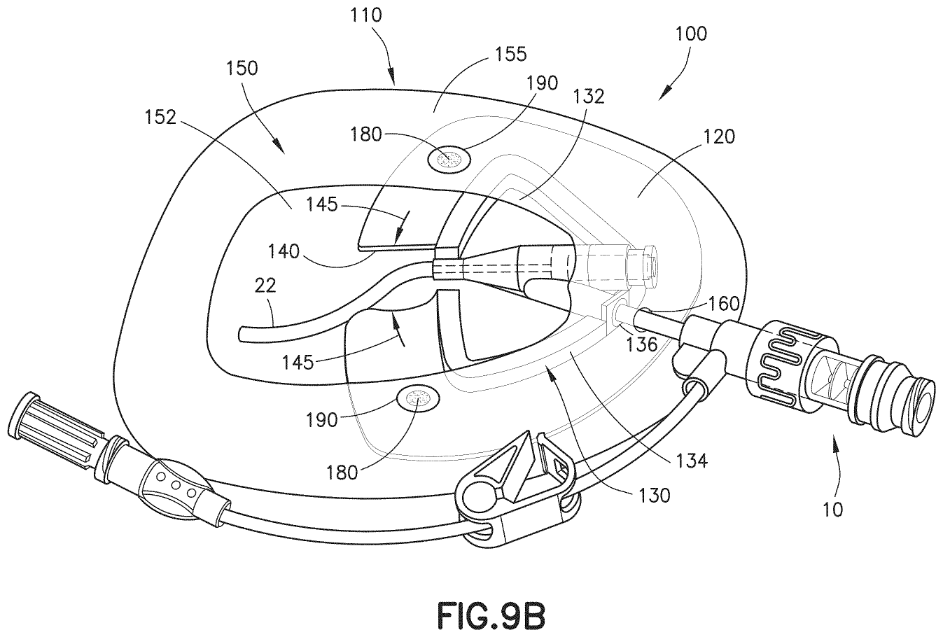

Turning to FIGS. 9A and 9B, in non-limiting embodiments, stabilization device 110 may include one or more positioning markers. In non-limiting embodiments, base dressing 120 may include one or more first positioning markers 180, arranged at one or more locations on base dressing 120. As with other markers disclosed herein, first positioning markers 180 may be any suitable indicia visible to a medical professional as stabilization device 110 is applied to a patient's skin. Top dressing 150 may include one or more corresponding second positioning markers 190. As with other markers disclosed herein, corresponding second positioning markers 190 may be any suitable indicia visible to a medical professional. In use, a medical professional may apply top dressing 150 to the patient's skin, and by lining up corresponding second positioning markers 190 such that they overlay the one or more first positioning markers 180 on base dressing 120, a medical user can be confident that top dressing 150 is applied correctly. Correct application of top dressing 150 may mean, in some embodiments, one or more openings and/or extensions 160 of top dressing 150 is properly positioned to allow a connector 30, port 18, and/or fluid conduit 24, 34 attached to or extending from catheter adapter 12 to pass through top dressing 150, and/or to allow top dressing 150 to be wrapped about a connector 30, port 18, and/or fluid conduit 24, 34 attached to or extending from catheter adapter 12.

Also provided herein is a method of stabilizing a catheter assembly 10. The method may include steps of inserting a catheter, such as a catheter 22 exemplified in FIG. 1, into a patient, using any known process. Once catheter 22 is inserted into the patient, any introducer(s) may be removed or withdrawn from catheter 22. A base dressing, such as a base dressing 120 described herein, can then be applied to the patient's skin, for example by removing a peelable backing covering the adhesive-containing lower surface and pressing the base dressing 120 to the patient's skin. In non-limiting embodiments, a support, such as support 130 described herein, may be provided as a separate element, and upper surface of base dressing 120 may also include a peelable backing covering an adhesive-containing upper surface. In non-limiting embodiments, one or more markers, such as alignment marker(s) 145 and/or maximum and minimum distance markers 147, 149, may be used to align and/or position base dressing 120 relative to insertion site of catheter 22. An adhesive or antimicrobial may then be applied at the site of insertion, and top dressing, such as top dressing 150 described herein, may be applied to the base dressing 120 and/or patient's skin, for example by removing a peelable backing covering a surface of top dressing 150 that includes an adhesive and pressing top dressing 150 to base dressing 120 and/or the patient's skin. In non-limiting embodiments, base dressing 120 includes one or more first positioning markers 180 as described herein, and top dressing 150 includes one or more corresponding second positioning markers 190, and application of top dressing 150 includes alignment of corresponding second positioning markers 190 to one or more first positioning markers 180 prior to pressing top dressing 150 to base dressing 120 and/or the patient's skin.

Although the present disclosure has been described in detail for the purpose of illustration based on what is currently considered to be the most practical and preferred embodiments or aspects, it is to be understood that such detail is solely for that purpose and that the present disclosure is not limited to the disclosed embodiments or aspects, but, on the contrary, is intended to cover modifications and equivalent arrangements that are within the spirit and scope of the appended claims. For example, it is to be understood that the present disclosure contemplates that, to the extent possible, one or more features of any embodiment may be combined with one or more features of any other embodiment.

The invention claimed is:

1. A stabilization device for a catheter, comprising:
a base defining a base perimeter, the base comprising a top surface and a bottom surface;
a support surface arranged on the top surface of the base, the support surface comprising a wall extending away from the top surface of the base and defining a support surface perimeter, the support surface perimeter having substantially the same shape as a catheter adapter; and
a dressing defining a dressing perimeter, the dressing configured to cover the base and the support surface.

2. The stabilization device of claim 1, wherein the wall comprises a foam material.

3. The stabilization device of claim 1, wherein the wall comprises a first opening configured to receive a component of a catheter adapter therein.

4. The stabilization device of claim 3, wherein the wall comprises a second opening configured to receive a component of the catheter adapter therein.

5. The stabilization device of claim 4, wherein the second opening is arranged at a 25 degree angle, 45 degree angle, 90 degree angle, and/or 135 degree angle relative to the first opening.

6. The stabilization device of claim 1, wherein the bottom surface of the base comprises an adhesive configured to releasably attach the base to skin of a patient onto which the stabilization device is applied.

7. The stabilization device of claim 1, wherein the support surface is angled relative to the base such that the catheter adapter received within the support surface perimeter is not parallel to the skin of a patient onto which the stabilization device is applied.

8. The stabilization device of claim 7, wherein the support surface comprises a foam.

9. The stabilization device of claim 7, wherein the support surface is angled from about 3 degrees to about 10 degrees relative to the skin of the patient onto which the stabilization device is applied.

10. The stabilization device of claim 1, wherein the dressing is substantially transparent.

11. The stabilization device of claim 1, wherein the base comprises a cutout configured to receive the catheter therein.

12. The stabilization device of claim 11, wherein the base comprises one or more alignment markers.

13. The stabilization device of claim 11, wherein the base comprises two alignment markers arranged on opposite sides of the cutout.

14. The stabilization device of claim 1, wherein the base comprises one or more distance markers representing a distance between an insertion site of the catheter and the catheter adapter received on the support surface.

15. The stabilization device of claim 1, wherein the base comprises one or more first positioning markers and the top dressing comprises one or more corresponding positioning markers, such that when the top dressing is applied, the one or more corresponding positioning markers overlay the one or more first positioning markers.

16. The stabilization device of claim 1, wherein the base comprises one or more second support surfaces.

17. The stabilization device of claim 16, wherein the one or more second support surfaces are positioned outside of the base perimeter.

18. A stabilized catheter set, comprising:
a catheter assembly comprising:
a catheter adapter, comprising:
a distal end;
a proximal end;
a lumen arranged between and in fluid communication with the distal end and the proximal end; and
a catheter arranged at the distal end of the catheter adapter and in fluid communication with the lumen; and
a stabilization device, the stabilization device comprising:
a base defining a base perimeter, the base including a top surface and a bottom surface, a support surface arranged on the top surface of the base, the support surface comprising a wall extending away from the top surface of the base and defining a support surface perimeter, the support surface perimeter having substantially the same shape as a catheter adapter, and
a dressing defining a dressing perimeter, the dressing configured to cover the base and the support surface.

19. The stabilized catheter set of claim 18, wherein the catheter adapter further comprises a port in fluid communication with the lumen.

20. The stabilized catheter set of claim 19, further comprising a fluid conduit having a distal end and a proximal end, the distal end coupled to and in fluid communication with the port.
